(19) United States
(12) Patent Application Publication
(10) Pub. No.: US 2023/0420692 A1
(43) Pub. Date: Dec. 28, 2023

(54) METHOD OF ALTERING DEGREE OF CURVATURE OF A MOLECULAR CATALYST FOR HIGHER CATALYTIC ACTIVITY

(71) Applicant: City University of Hong Kong, Hong Kong (HK)

(72) Inventors: Ruquan YE, Hong Kong (HK); Jianjun SU, Hong Kong (HK)

(21) Appl. No.: 18/316,201

(22) Filed: May 11, 2023

Related U.S. Application Data
(60) Provisional application No. 63/355,662, filed on Jun. 27, 2022.

(51) Int. Cl. H01M 4/90; C07C 29/156; H01M 8/08

(52) U.S. Cl. CPC H01M 4/9083; C07C 29/156; H01M 4/9008; H01M 8/08; B82Y 30/00

(57) ABSTRACT

A method of altering degree of curvature of a molecular catalyst for CO₂ reduction reaction (CO₂RR). Briefly, providing a single-walled carbon nanotube (SWCNT). Next, a molecular catalyst having active sites for CO₂RR is provided. The molecular catalyst is dispersed on the SWCNT. A curvature of the active sites of the molecular catalyst is then induced. The alternation of degree of curvature of a molecular catalyst is beneficial for higher catalytic activity in transforming CO₂ into methanol.

### CROSS-REFERENCE TO RELATED APPLICATIONS

The present application claims priority from U.S. provisional patent application Ser. No. 63/355,662 filed Jun. 27, 2022, and the disclosure of which is incorporated herein by reference in its entirety.

### FIELD OF THE INVENTION

The invention relates to the technical field of catalysts. In particular, it relates to an alternation of a degree of curvature of a molecular catalyst for higher catalytic activity.

### BACKGROUND OF THE INVENTION

The electrochemical conversion of CO₂ is a promising method for producing chemicals and plays a crucial role in carbon recycling and storing renewable electricity in a convenient and high energy-density form. Although many studies have achieved industrial-scale conversion of CO₂-to-CO with high CO selectivity (over 90 %) and current density (larger than 200 mA cm⁻²), most reactions are limited to a two-electron reduction process that produces carbon monoxide or formate (HCOO⁻). However, reducing CO₂ beyond two electrons on an industrial scale remains a significant challenge.

Methanol (MeOH) is a versatile one-carbon (C1) product and widely used green fuel with an energy density of 15.6 MJ/L. It is also an extremely important intermediate for the production of useful chemicals and fuels such as dimethyl ether (CH₃OCH₃, DME) and methyl tert-butyl ether (CH₃-OC(CH₃)₃, MTBE). Methanol has advantages over hydrogen as it can be stored at atmospheric pressure and directly used in internal combustion engines due to its high-octane rating. Methanol can also be added into fuel cells. However, the carbon dioxide reduction reaction (CO₂RR) to methanol through a six-electron reduction pathway is still in an early stage of development. Although copper has been shown to catalyze the CO₂RR by a multi-electron pathway, the copper induced reaction usually results in a mixture of products that require an extensive separation process.

In 1984, a study of molecular catalyst using cobalt phthalocyanine (CoPc) showed a methanol Faradaic efficiency (FE) of less than 5%, but this low FE number was not given much attention until more recently. Research groups, led by Wang and Robert, respectively reported improved methanol FE when CoPc is deposited on multiwalled carbon nanotubes (MWCNT). This improvement is attributed to the dispersion of CoPc on the conductive substrate. MWCNTs have since been widely used with different catalysts, but the methanol production rate remains marginal (Wang et al., Nature 575, 639-642, 2019) (Robert et al., Angew. Chemie Int. Ed. 58, 16172-16176, 2019). Questions have arisen about the underlying mechanism of the CoPc/MWCNT activity. Conventional methods for tuning the activities of molecular catalysts require the design of new structures or change to functional groups, which can be time-consuming and costly.

There is a need to create a more efficient reaction pathway for catalytic activity without complex chemical modification. In particular, the industry is searching for a solution for a solution that can yield a high amount of methanol from the CO₂RR in the six-electron reduction pathway.

### SUMMARY OF THE INVENTION:

This section is for the purpose of summarizing some aspects of embodiments of the invention and to briefly introduce some further embodiments. In this section, as well as in the abstract and the title of the invention of this application, simplifications or omissions may be made to avoid obscuring the purpose of the section, the abstract and the title, and such simplifications or omissions are not intended to limit the scope of the invention.

The present invention has been made in view of the above-mentioned problems of the need of a more efficient reaction pathway for catalytic activity without complex chemical modification. The structure of the catalyst is finely tuned to alter the morphology of its active site. A catalyst having a substantially flat structure undergoes distortion to an extent that its active site is bent, or a curvature is created by the distortion. This distortion will have an effect on the bonding strength between the active site and the target molecule. The curvature of the target molecule can be manipulated by single wall carbon nanotube (SWCNT). The distorted target molecule exhibits a higher Faraday efficiency value in the catalytic process.

In accordance with a first aspect of the present invention, the present invention provides a method of altering degree of curvature of a molecular catalyst for CO₂ reduction reaction (CO₂RR) for higher catalytic activity. The method includes providing a single-walled carbon nanotube (SWCNT). Next, a molecular catalyst having active sites for CO₂ reduction reaction is provided. The molecular catalyst is dispersed on the SWCNT. A curvature of the active sites of the molecular catalyst is then induced.

In a further embodiment of the present invention, dispersing the molecular catalyst on the SWCNT includes providing a solution having N,N-dimethylformamide. The molecular catalyst and the SWCNT are then added into the solution. A sonication is performed to the solution. A magnetic stirring is performed to the solution.

In a further embodiment of the present invention, the inducing the curvature of the active sites of the molecular catalyst by the SWCNT includes initiating a non-parallel π-π interaction between the molecular catalyst and the SWCNT.

In a further embodiment of the present invention, the inducing the curvature of the active sites of the molecular catalyst by the SWCNT includes bending the active sites of the molecular catalyst from a flat configuration to a curved configuration.

In a further embodiment of the present invention, after the inducing the curvature of the active sites of the molecular catalyst by the SWCNT further includes receiving CO₂ at the active site, releasing CO from the active site, and releasing methanol from the active sites.

In a further embodiment of the present invention, the SWCNT is smaller in size than the molecular catalyst. In another embodiment of the present invention, the SWCNT is bigger in size than the molecular catalyst.

In a further embodiment of the present invention, the SWCNT has a diameter of at least 1 nm.

In a further embodiment of the present invention, the SWCNT has a diameter between 1 and 6 nm.

In a further embodiment of the present invention, the molecular catalyst is cobalt phthalocyanine.

In a further embodiment of the present invention, the molecular catalyst is nickel phthalocyanine.

In accordance with a second aspect of the present invention, the present invention provides a method of altering degree of curvature of a molecular catalyst including providing a single-walled carbon nanotube (SWCNT) and providing a molecular catalyst. The molecular catalyst is dispersed on the SWCNT. A curvature of the active sites of the molecular catalyst is induced by the SWCNT.

In a further embodiment of the present invention, the molecular catalyst is iron phthalocyanine.

In a further embodiment of the present invention, after inducing the curvature of the active sites of the molecular catalyst by the SWCNT further includes effecting an oxygen reduction reaction by the molecular catalyst.

In a further embodiment of the present invention, the oxygen reduction reaction is a four-electron process.

In a further embodiment of the present invention, the inducing the curvature of the active sites of the molecular catalyst includes bending the active sites of the molecular catalyst from a flat configuration to a curved configuration.

In a further embodiment of the present invention, the SWCNT is smaller in size than the molecular catalyst. In another embodiment of the present invention, the SWCNT is bigger in size than the molecular catalyst.

In accordance with a third aspect of the present invention, the present invention provides a molecular catalyst having an altered degree of curvature, more particularly, the molecular catalyst is dispersed on a single-walled carbon nanotube (SWCNT) for altering degree of curvature.

In a further embodiment of the present invention, the molecular catalyst having active sites for CO₂ reduction reaction (CO₂RR).

In a further embodiment of the present invention, the SWCNT has a diameter of 1-6 nm.

In a further embodiment of the present invention, the altered degree of curvature ranges from 1 to 96 degree.

In accordance with a fourth aspect of the present invention, the present invention provides a fuel cell with a molecular catalyst having active sites for oxygen reduction reaction.

### BRIEF DESCRIPTION OF THE DRAWINGS

In order to more clearly illustrate the technical solutions of the embodiments of the present invention, the drawings needed to be used in the description of the embodiments will be briefly introduced below. It is obvious that the drawings in the following description are only some embodiments of the present invention, and it is obvious for those skilled in the art to obtain other drawings based on these drawings without inventive exercise, in which:

FIGS. 1A and 1B are transmission electron microscopy (TEM) images showing a morphology of CoPc/SWCNT and SWCNT respectively;

FIG. 2 is a schematic diagram showing a curvature of a molecular catalyst according to an embodiment of the present invention;

FIG. 3 is a schematic diagram showing a curvature of a molecular catalyst according to an embodiment of the present invention;

FIG. 4 is a graph showing UV-Vis absorption profiles of CoPc, CoPc/MWCNT, and CoPc/SWCNT according to an embodiment of the present invention;

FIG. 5 is a graph showing Raman spectrum of CoPc, SWCNT, MWCNT, CoPc/MWCNT, and CoPc/SWCNT according to an embodiment of the present invention;

FIG. 6 is a graph showing the cyclic voltammetry readouts of different samples in CO₂RR in 0.5 M KHCO3;

FIG. 7 is a graph showing methanol partial current density of CoPc/MWCNT and CoPc/SWCNT;

FIGS. 8A and 8B are graphs showing methanol Faradaic efficiency as a function of potential in CO₂RR of CoPc/SWCNT and CoPc/MWCNT respectively;

FIG. 9 is a graph showing the Faradaic efficiency of CoPc/MWCNT and CoPc/SWCNT in CO₂RR in flow cell configuration (1M KOH solution);

FIG. 10 is a graph showing total and methanol partial current density of CoPc/MWCNT and CoPc/SWCNT in CO 2 RR in flow cell configuration (1M KOH solution);

FIG. 11 is a graph showing the electrochemical ORR performance (0.1M HOH) of FePc/SWCNT, FePc/MWCNT, and FePc/50;

FIG. 12 is a graph showing CO₂RR performance in H-cell (0.5M KHCO3) of NiPc/SWCNT, NiPc/SWCNT and NiPc/50;

FIGS. 13A and 13B are TEM images of CoPc/MWCNT and MWCNT respectively;

FIGS. 14A and 14B are readouts of Raman spectrum of different samples confirming the deposition of CoPc on MWCNT and SWCNT; FIG. 14A shows the Raman spectrum of CoPc/MWCNT, CoPc/SWCNT and CoPc; and FIG. 14B exhibits the Raman spectrum of MWCNT and SWCNT;

FIG. 15 is an UV-VIS readouts graph showing MWCNT and SWCNT are able to induce the change of molecular orbitals;

FIGS. 16A and 16B depict chronoamperometric study (current density as a function of time) of CoPc/MWCNT and CoPc/SWCNT using H-cell in 0.5M KHCO₃ respectively;

FIGS. 17A and 17B are graphs showing CV traces for SO₄²⁻adsorption of different samples;

FIGS. 17C and 17D are graphs showing CV traces for hydroxide adsorption in 0.1M NaOH solution of different samples;

FIGS. 18A and 18B are cyclic voltammograms graphs showing SWCNT and MWCNT have different effects on tuning molecular catalytic activity evaluated by screening different samples in 0.5 M aqueous KHCO₃ solution under Ar;

FIG. 19 depicts the chronoamperometric curves of different samples using H-cell in 0.5 M KHCO₃ at −0.9V vs. RHE;

FIG. 20 shows the NMR results of CoPc, MWCNT, and SWCNT using H-cell in 0.5 M KHCO₃ at −0.9V vs. RHE;

FIG. 21 is a graph showing chronoamperometric curves of the samples with different CoPc to SWCNT mass ratio using H-cell in 0.5 M KHCO₃ at −0.9V vs. RHE;

FIG. 22 is a graph showing Faradaic efficiency of samples with different CoPc to SWCNT mass ratio using H-cell in 0.5 M KHCO₃ at −0.9V vs. RHE;

FIG. 23 is a Faradaic efficiency of samples having different CNT diameters using different samples using H-cell in 0.5 M KHCO₃ at −0.9V vs. RHE;

FIG. 24 is a methanol partial current density of samples having different CNT diameters using different samples using H-cell in 0.5 M KHCO₃ at −0.9V vs. RHE; and

FIG. 25 is chronoamperometric curves of samples having different CNT diameters using different samples using H-cell in 0.5 M KHCO₃ at −0.9V vs. RHE.

### DETAILED DESCRIPTION

In order to make the aforementioned objects, features and advantages of the present invention comprehensible, embodiments accompanied with figures are described in detail below. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

In accordance with a first aspect of the present invention, a method of

altering degree of curvature of a molecular catalyst for CO₂ reduction reaction (CO₂RR) for higher catalytic activity is provided. Briefly, a single-walled carbon nanotube (SWCNT) is first provided. Next, a molecular catalyst having active sites for CO₂RR is also provided. The molecular catalyst is dispersed on the SWCNT. A curvature of the active sites of the molecular catalyst is then induced.

In one embodiment, the dispersing the molecular catalyst on the SWCNT includes providing a solution having N,N-dimethylformamide; adding the molecular catalyst and the SWCNT into the solution; performing a sonication to the solution; and performing a magnetic stirring to the solution.

In one embodiment, the inducing the curvature of the active sites of the molecular catalyst includes initiating a non-parallel π-π it interactions between the molecular catalyst and the SWCNT.

In one embodiment, the inducing the curvature of the active sites of the molecular catalyst includes bending the active sites of the molecular catalyst from a flat configuration to a curved configuration.

In one embodiment, after the inducing the curvature of the active sites of the molecular catalyst, it further includes receiving CO₂ at the active site, releasing CO from the active site, and releasing methanol from the active sites.

In one embodiment, the SWCNT is smaller in size than the molecular catalyst. In particular, the diameter of SWCNT is less than the size of the molecular catalyst.

In another embodiment of the present invention, the SWCNT is bigger in size than the molecular catalyst.

In one embodiment, the SWCNT has a diameter of at least 1 nm.

In another embodiment, the SWCNT has a diameter of 1-6 nm.

In one embodiment, the molecular catalyst is cobalt phthalocyanine. In another embodiment, the molecular catalyst is nickel phthalocyanine.

In accordance with a second aspect of the present invention, a method of altering degree of curvature of a molecular catalyst is provided. The method includes the following steps: providing a single-walled carbon nanotube (SWCNT); providing a molecular catalyst; dispersing the molecular catalyst on the SWCNT; and inducing a curvature of the active sites of the molecular catalyst.

In one embodiment, the molecular catalyst is iron phthalocyanine.

In one embodiment, after inducing the curvature of the active sites of the molecular catalyst, it further includes effecting an oxygen reduction reaction by the molecular catalyst.

In one embodiment, the oxygen reduction reaction is a four-electron process.

In one embodiment, the inducing the curvature of the active sites of the molecular catalyst includes bending the active sites of the molecular catalyst from a flat configuration to a curved configuration.

In one embodiment, the SWCNT is smaller in size than the molecular catalyst. In another embodiment of the present invention, the SWCNT is bigger in size than the molecular catalyst.

In accordance with a third aspect of the present invention, the present invention provides a molecular catalyst having an altered degree of curvature, more particularly, the molecular catalyst is dispersed on a single-walled carbon nanotube (SWCNT) for altering degree of curvature.

In one embodiment, the molecular catalyst having active sites for CO₂ reduction reaction (CO₂RR).

In one embodiment, the altered degree of curvature ranges from 1 to 96 degree.

In one embodiment, the SWCNT has a diameter of 1-6 nm.

In accordance with a fourth aspect of the present invention, the present invention provides a fuel cell with a molecular catalyst having active sites for oxygen reduction reaction.

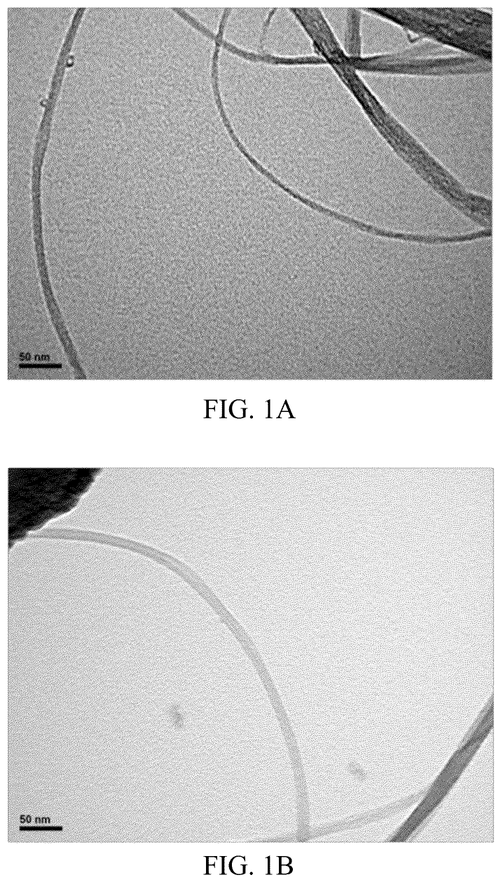

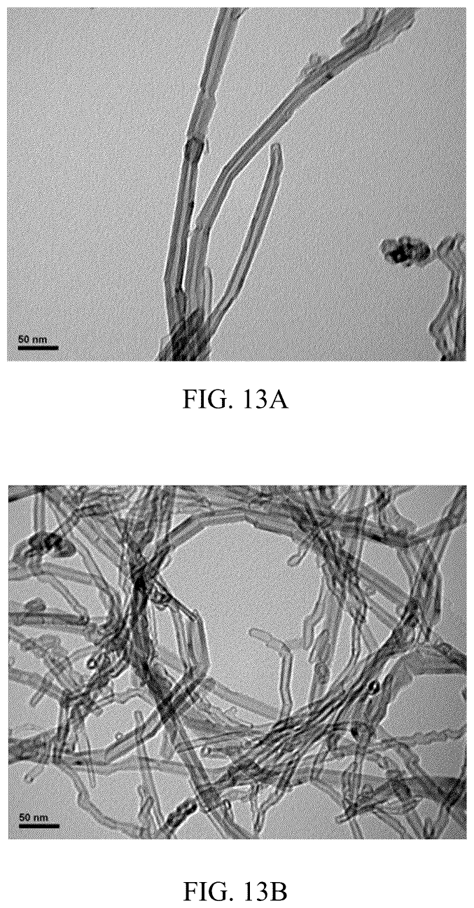

The molecular catalyst for CO₂ reduction reaction (CO₂RR) is prepared by adding the molecular catalyst and the single-walled carbon nanotube (SWCNT) into the N,N-dimethyl formamide (DMF) solution, followed by sonication and magnetic stirring. In one embodiment, the molecular catalyst used is cobalt phthalocyanine (CoPc). A comparison sample is prepared using the same method, but by adding the molecular catalyst and multi-walled carbon nanotube (MWCNT) to the DMF solution. The transmission electron microscopy (TEM) images, as shown in FIGS. 1A and 1B, indicate that the morphology of the CoPc/SWCNT hybrid, shown in FIG. 1A, and the bare SWCNT, shown in FIG. 1B, are substantially identical. No aggregated CoPc lumps are clung to the SWCNT strips. However, FIG. 13A shows that aggregated CoPc lumps are observed on the CoPc/MWCNT hybrid, while FIG. 13B shows the bare MWCNT strips.

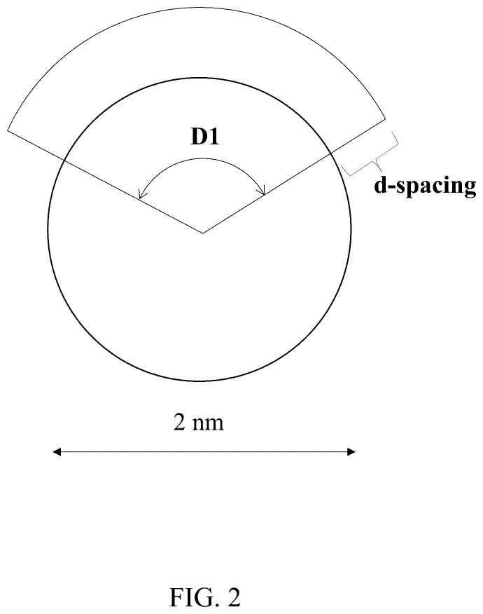

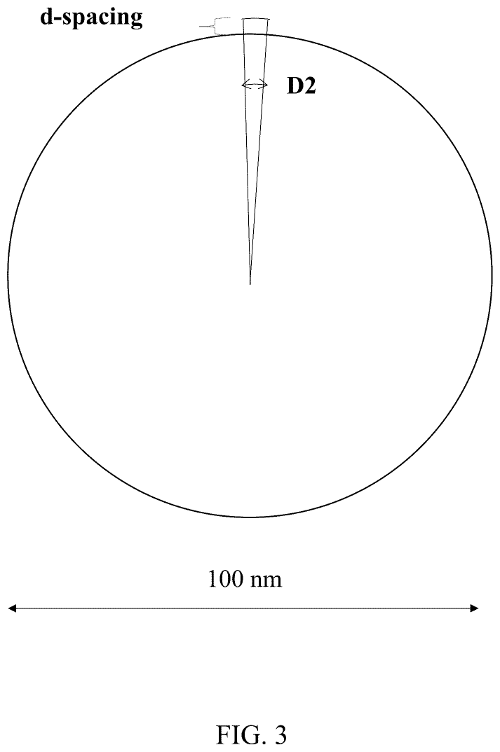

The SWCNT has a diameter that is smaller than the size of the molecular catalyst. For example, a CoPc molecule has a side length of ca. 1.24 nm, while the SWCNT has a diameter of approximately 2 nm. The curvature of the molecular catalyst is initiated by the interactions between the SWCNT and the molecular catalyst through the nonparallel π-π interactions. Referring to FIG. 2, a schematic diagram showing the induced curvature of the molecular catalyst. D1 is designated as the degree of curvature of the molecular catalyst. The round circle represents the SWCNT, and the length below it indicates the diameter of the SWCNT. The diameter of the SWCNT in FIG. 2 is approximately 2 nm, and the molecular catalyst has a diameter of approximately 2 nm. The interlayer distance between the SWCNT and the molecular catalyst is defined as d-spacing. While the d-spacing is approximately 0.3 nm, the degree of curvature D1 is approximately 100°. Referring to FIG. 3, another schematic diagram showing the induced curvature of the molecular catalyst. The diameter of the SWCNT is increased to approximately 100 nm, and the molecular catalyst has the same diameter of approximately 2 nm. While the d-spacing is also set as approximately 0.3 nm, the degree of curvature D2 is altered to approximately 2°. In other words, the curvature of the molecular catalyst can be finely tuned by adjusting the dimension of the SWCNT. A smaller SWCNT in diameter will induce a larger distortion (curvature) from flat to bent on the molecular catalyst, whereas a larger SWCNT in diameter has a less bending impact on the molecular catalyst.

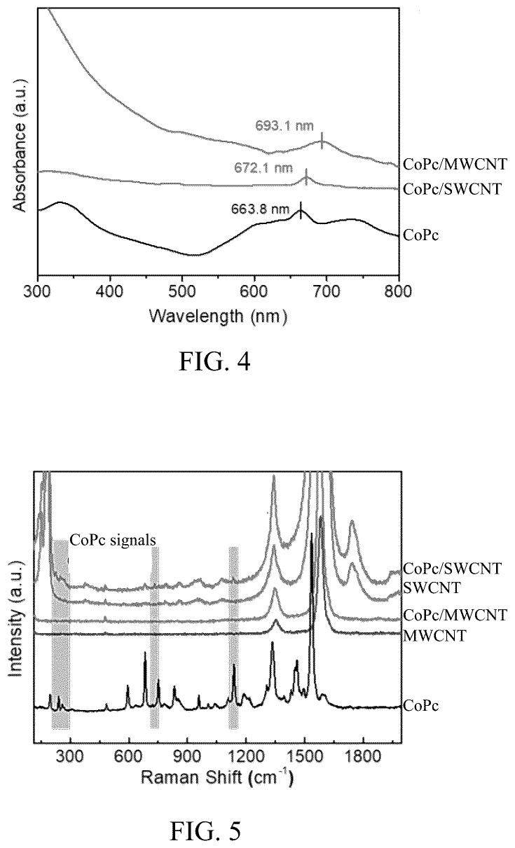

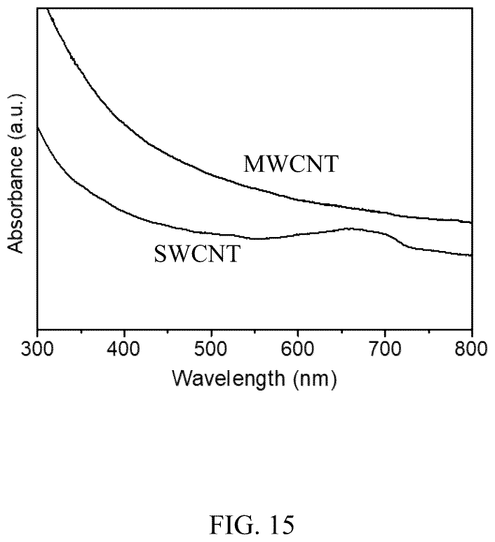

Referring to FIG. 4, the UV-vis spectra of different molecular catalyst hybrids show the Q band of the CoPc molecules. There is a shift from 693.1 nm of CoPc/MWCNT to 672.1 nm of CoPc/SWCNT. The shift of peak position indicates the change of molecular orbitals due to the molecular curvature induced by the strong interaction with the CNT support. Further, the UV-vis spectra of MWCNT and SWCNT are also shown in FIG. 15.

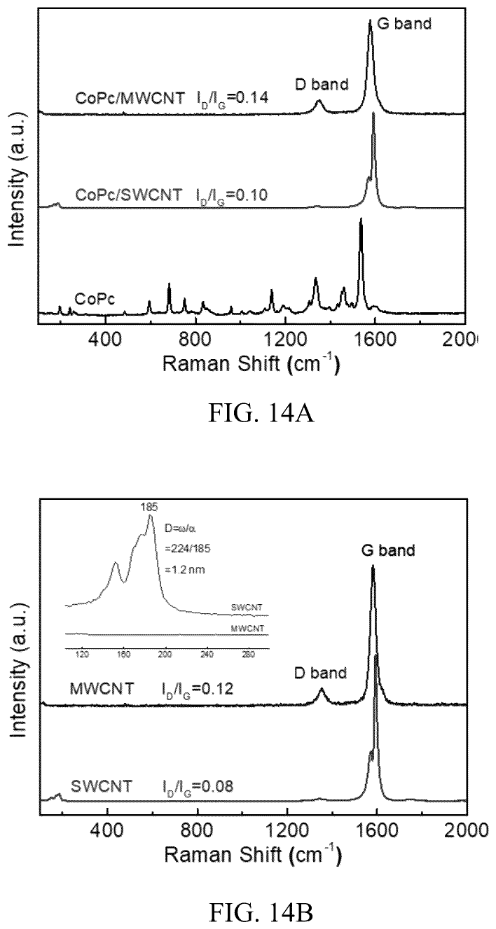

Referring to FIG. 5, Raman spectrum of CoPc, SWCNT, MWCNT, CoPc/MWCNT, and CoPc/SWCNT are shown. The shaded areas indicate the signals of molecular catalytic activities. More specifically, the molecular catalytic activities observed in CoPc are also found in CoPc/SWCNT hybrid and CoPc/MWCNT hybrid. Further, FIG. 14A shows the Raman spectrum of CoPc/MWCNT, CoPc/SWCNT and CoPc, while FIG. 14B shows the Raman spectrum of MWCTN and SWCNT simply.

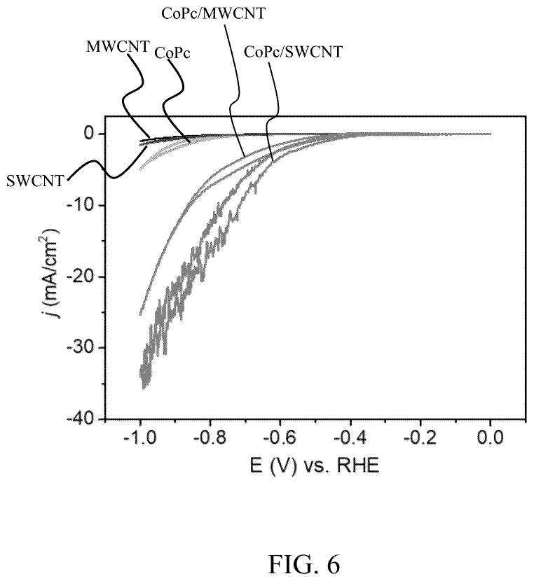

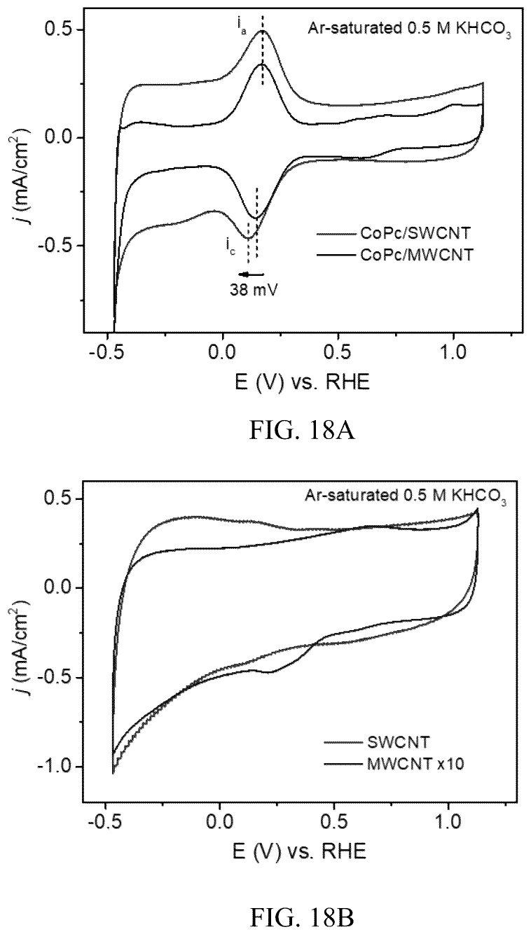

The electrochemical CO₂ reduction reaction (CO₂RR) is carried out in a customized glass H-cell with continuous CO₂ saturated in 0.5M KHCO₃ at 3 sccm flow rate. Turning to FIG. 6, results of cyclic voltammograms (CV) of different samples in CO₂-saturated electrolyte are shown. The curve of CoPc/SWCNT is more pronounced in comparison with the curve of CoPc/MWCNT or CoPc alone. This result suggests that CoPc/SWCNT has much higher catalytic activity rate. Furthermore, FIGS. 18A and 18B indicate that the cyclic voltammograms of CoPc/SWCNT and CoPc/MWCNT, respectively. The cyclic voltammograms show the shifting of redox potential, indicating that the CoPc' s electrochemical properties can be affected by the CNTs.

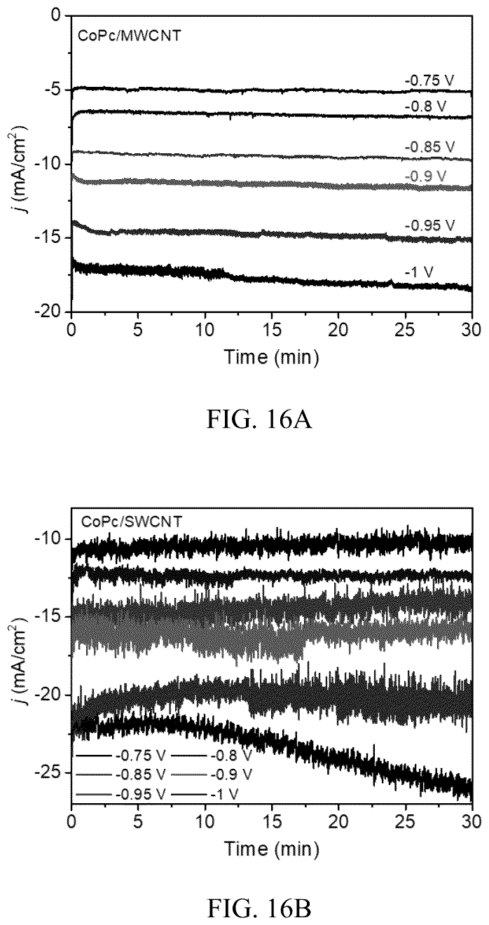

Referring to FIGS. 16A and 16B, the CO₂RR selectivity of CoPc/SWCNT and CoPc/MWCNT is assessed under different potentials by chronoamperometry tests, nuclear magnetic resonance (NMR) and gas chromatography (GC). The liquid and gas products in the reaction are analyzed respectively.

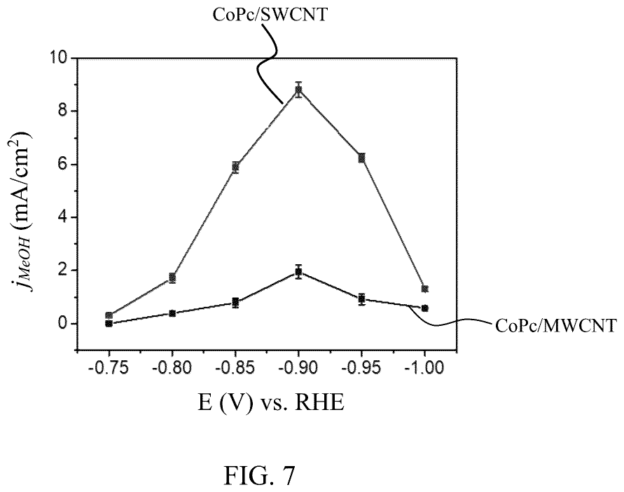

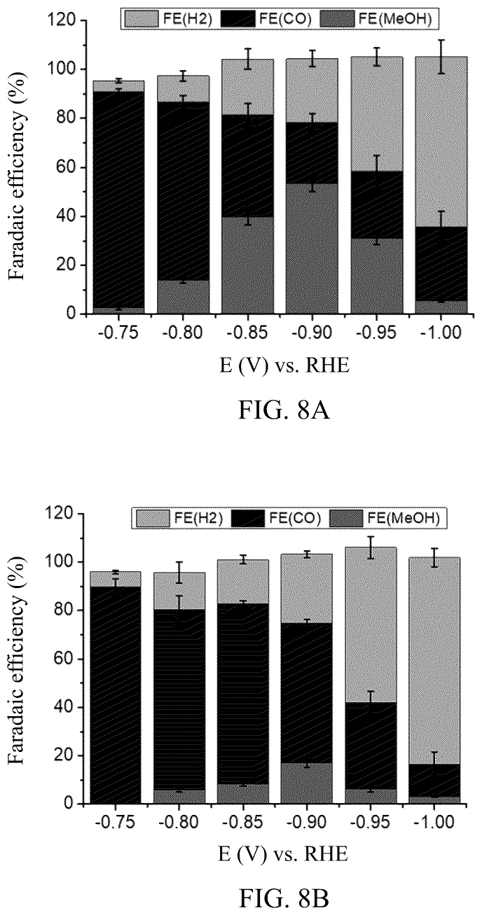

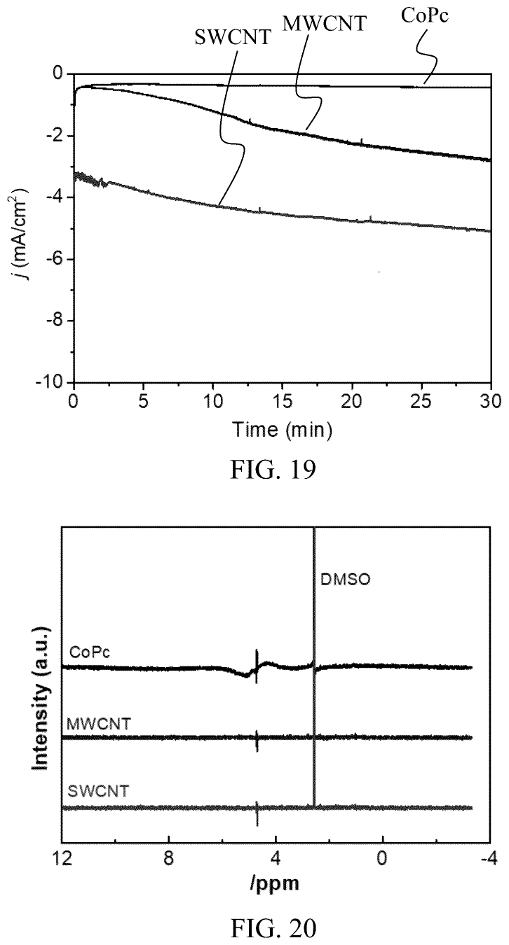

Referring to FIGS. 8A and 8B, a volcano-shaped Faradaic efficiency (FE) methanol (MeOH) dependence test is performed at the applied potentials from −0.75 to −1 V vs RHE. As shown in FIG. 8A, at −0.9 V, a maximum Me0H FE of 53.2% is achieved by CoPc/SWCNT, whereas, as shown in FIG. 8B, at −0.9V, CoPc/MWCNT achieves Me0H FE of merely 16.8%. Turning to FIG. 7, MeOH partial current density of CoPc/SWCNT and CoPc/MWCNT is shown. CoPc/SWCNT reaches a maximum MeOH partial current density of 8.8 mA cm-2 at −0.9 V. In contrast, turning to FIGS. 19 and 20, CoPc, bare SWCNT, and MWCNT show negligible electrocatalytic CO₂RR with marginal CO product under the same applied potentials.

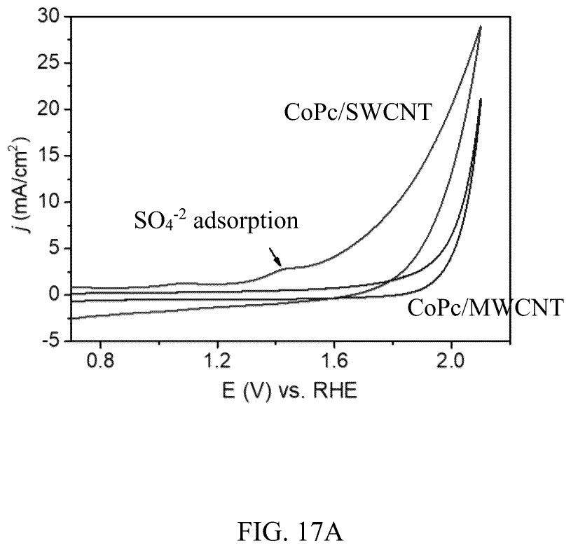

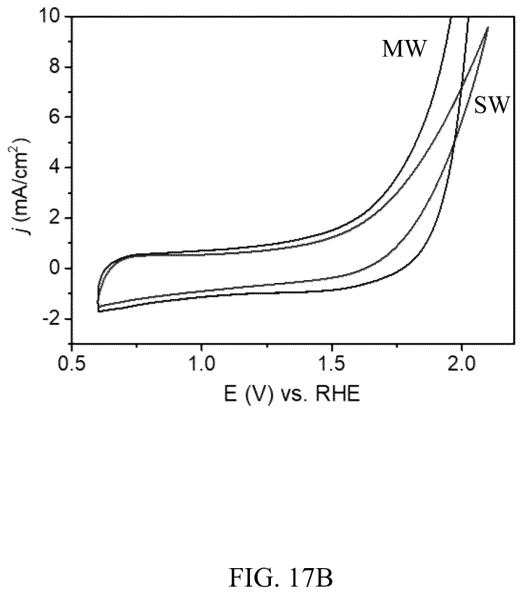

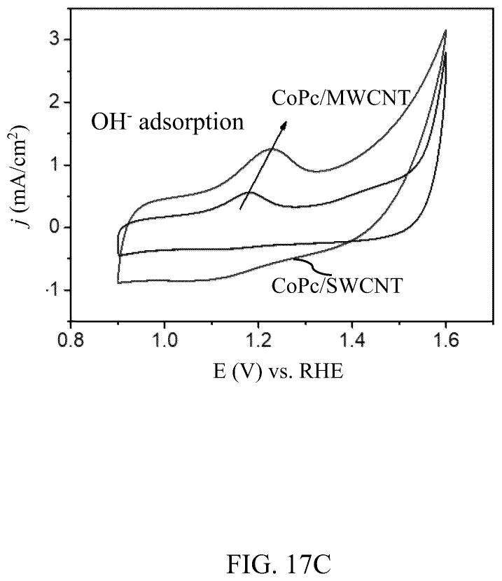

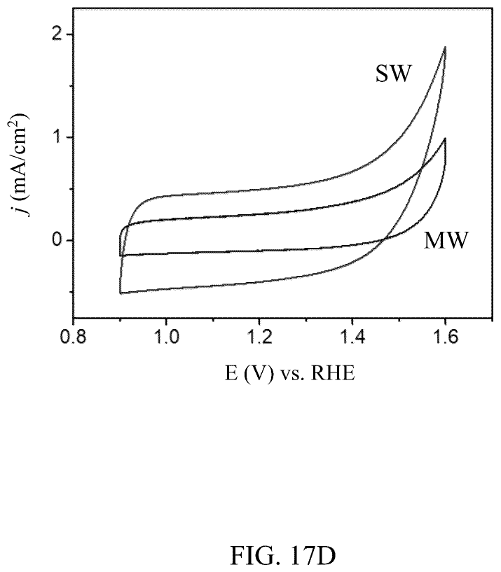

CoPc/SWCNT is further analyzed by density functional theory (DFT) calculations on the basis of the model of EXAFS fitting results. The stabilization of CO₂^{⋅-}on the surfaces of the molecular catalysts plays an important role in CO₂RR. The overpotential of SO₄²⁻adsorption over the samples can be used to measure the binding strength of the intermediate CO₂^{⋅-}on the molecular catalyst surface during CO₂RR. SO₄²⁻adsorption of SWCNT, MWCNT, CoPc/SWCNT and CoPc/MWCNT is studied by measuring the CV scans from 0.6 to 2.1 V (vs RHE) at 50 mV/s in 0.1 M H₂SO₄ electrolyte solution, as shown in FIGS. 17A and 17B. The results show that the peak of SO₄²⁻adsorption over CoPc/SWCNT is higher than that over CoPc/MWCNT. It suggests a stronger binding of SO₄²⁻over CoPc/SWCNT compared to CoPc/MWCNT.

The OH binding strength at the surface of the electrode is measured over SWCNT, MWCNT, CoPc/SWCNT and CoPc/MWCNT with the CV scans from 0.9 to 1.6 V (vs RHE) at 50 mV/s in the 0.1 M NaOH electrolyte solution, as shown in FIG. 17C and 17D. From the CV traces it can be told that the *—OH adsorption over the CoPc/SWCNT has higher onset potential compared to CoPc/MWCNT. It suggests a weaker binding strength of *—OH and *—OCH₃ over CoPc/SWCNT. That is, CoPc/SWCNT has the advantageous character of the desorption of *—OCH₃ from the surface of the electrode, thus resulting in higher selectivity toward MeOH formation by CoPc/SWCNT.

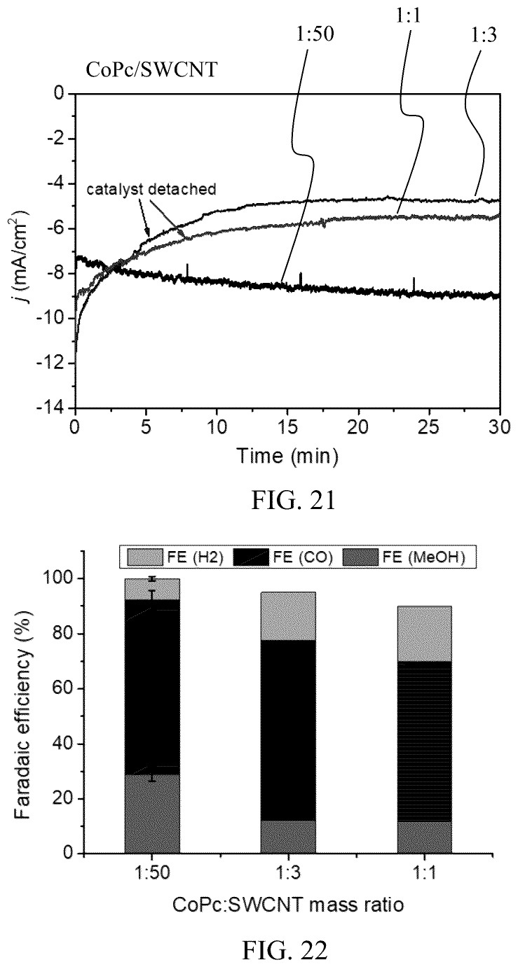

Referring to FIGS. 21 and 22, samples loaded with different amount of CoPc on SWCNT are tested. It is noted that the CoPc/SWCNT sample are in different CoPc to SWCNT mass ratios including 1:3, 1:1, and 1:50. Compared with the CoPc to SWCNT mass ratio of 1:10 having a MeOH FE of around 53.2%, increased CoPc to SWCNT mass ratio to 1:3 or 1:1 does not lead to a linear increase of MeOH FE. This result may be explained by the intermolecular architecture. The high loadings may increase the complexity in molecular stacking and decrease the catalyst utilization ratio. In addition, high CoPc loadings is likely to cause leaching of leaching of catalyst in the chronoamperometric test.

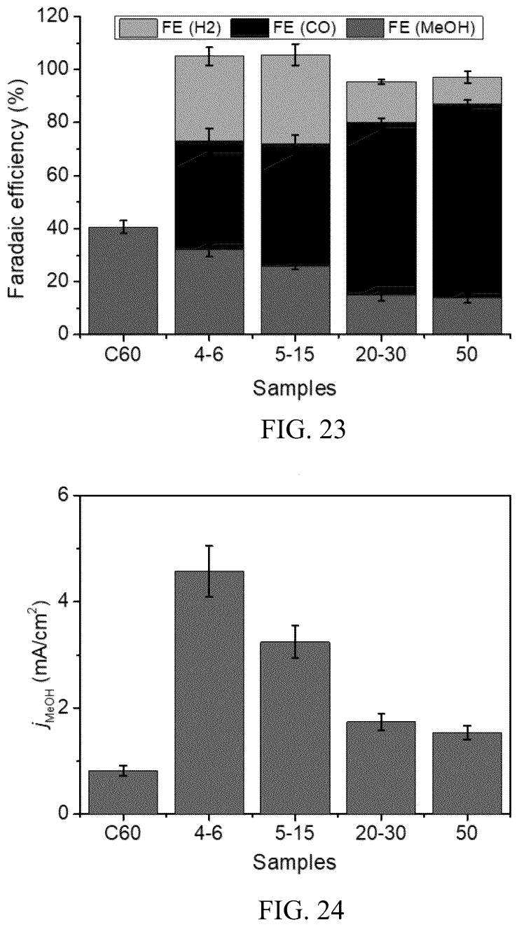

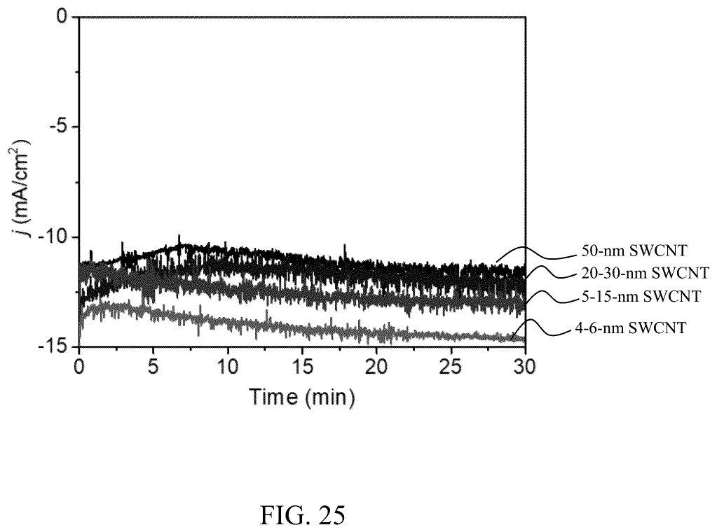

Referring to FIGS. 23-25, molecular catalysts are made with SWCNT having different diameters of 4-6, 5-15, 20-30, and 50 nm respectively. The MeOH FE of the different samples are carried out at −0.9 V MeOH. MeOH FE decreases from 53.2% of to 13.2% of as the diameter of the SWCNT increases. C₆₀ having a diameter of 1.1 nm also shows improvement in the CO₂-to-methanol conversion with a 40.6% selectivity.

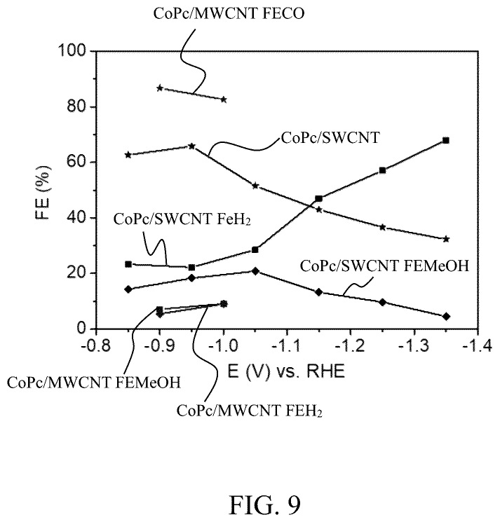

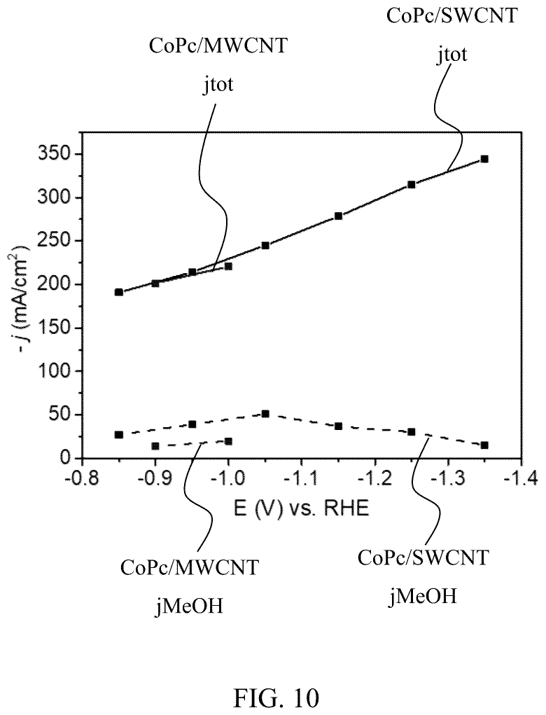

A higher current density is achieved by minimizing the CO₂ mass transport issue in the H-cell. An electrochemical microflow with gas diffusion electrode (GDE) configuration has been employed. In the flow cell configuration, 1M KOH medium instead of KHCO₃ is used as an electrolyte to improve ionic mobility and obtain better current density. The total current density in the flow cell increases to approximately 200-350 mA/cm². It is about 7 times more than that in the H-cell condition. Referring to FIGS. 9 and 10, the maximum observed FE for the main product (methanol: 20.8%) in the case of CoPc/SWCNT with a current density of 51.0 mA/cm² has been achieved.

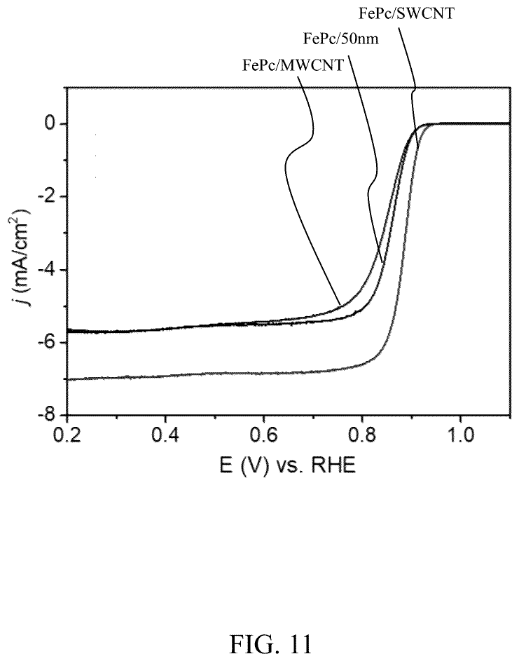

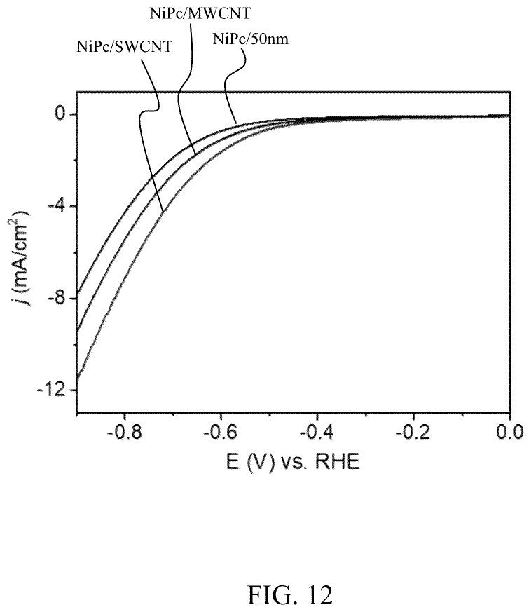

The curvature induced by SWCNT in molecular catalyst is tested in other systems. The oxygen reduction reaction (ORR) activity of FePc/SWCNT is investigated in O₂-saturated 0.1 M KOH with rotating disk electrode (RDE) measurement. Turning to FIG. 11, the results from the linear sweep voltammetry (LSV) are shown. FePc/SWCNT has higher catalytic activity in terms of the highest positive onset (Eₒₙₛₑₜ) and E_{1/2}. The E_{1/2} of FePc/SWCNT is 0.89 V, which is 30 mV more than that of FePc/MWCNT. Referring to FIG. 12, it is shown that NiPc/SWCNT has the highest CO₂RR performance in comparison with NiPc/MWCNT and NiPc/50. The current density of the NiPc/SWCNT reached −11.6 mA/cm², while it is −9.4 mA/cm² for NiPc/MWCNT and −7.8 mA/cm² for NiPc/50.

The alternation of molecular catalyst curvature can upregulate the activity at the active sites. The highly curved molecular catalyst undergoes severe distortion to release strain. This distortion has effects on the binding affinity between the molecular catalyst and its target upon receiving and releasing, for example, in the multi-electron transfer in CO₂RR. A distorted CoPc/SWCNT exhibits a 3.2-fold improvement in Faraday efficiency (FE) of Me0H compared to flat CoPc/MWCNT.

By monodispersing CoPc on SWCNT tailors the activity of molecular catalysts with significantly higher methanol selectivity. In contrast, a higher loading of CoPc or a larger-diameter SWCNT degrades the CO 2 -to-methanol conversion. X-ray spectroscopies combined with theoretical calculations suggest that the strong catalyst/support interaction induces molecular curvature and modulates the electronic structure of CoPc, leading to a balanced transition in receiving and releasing CO₂ rather than a CO desorption. A flow electrolyzer using CoPc/SWCNT as the catalyst achieves high FE for MeOH. Other supports such as using C₆₀ also improves the CO₂-to-methanol conversion with high selectivity.

### EXAMPLES

### Materials and Synthesis

CNT (XFNANO, Co., Ltd.) was pretreated in 6 mol L⁻¹ HCl solution for 12 h to remove any impurities. After that, the CNT sample was filtrated, washed with ultra-pure water and freeze-dried.

20 mg of the purified CNTs was subsequently dispersed in 20 ml of DMF using sonication. Then, an appropriate amount of CoPc dissolved in 5 ml DMF was added to the CNT suspension. The mixture was sonicated for 30 min to obtain a well-mixed suspension, which was further stirred at room temperature for 24 h. Subsequently, the mixture was centrifuged, and the precipitate was washed with DMF, ethanol and DI water. Finally, the precipitate was lyophilized to yield the final product. The samples with CNT substrates of different diameters SWCNT, MWCNT, 4-6 nm, 5-15 nm, 20-30 nm and >50 nm were denoted as CoPc/SWCNT, CoPc/MWCNT, 4-6, 5-15, 20-30, 50 respectively.

### Material Characterization

ICP-atomic emission spectroscopy (ICP-OED) measurements were conducted on Optima 8000 spectrometer. Samples were digested in hot concentrated HNO₃ for 1 h and diluted to desired concentrations. UV-vis spectrum was performed on a Shimadzu 1700 spectrophotometer in ethanol solution with a concentration of 1×10-5 mol/mL. The X-ray photoelectron spectroscopy data were collected on a Thermo ESCALAB 250Xi spectrometer equipped with a monochromatic AlK radiation source (1486.6 eV, pass energy 20.0 eV). The data were calibrated with C 1s 284.8 eV.

### Working Electrode Preparation

In H-cell, catalyst ink was prepared by dispersing 2 mg of catalyst in 1 mL of ethanol with 20 μL 5 wt. % Nafion solution (Sigma Aldrich, Nafion 117, 5 wt. %) and sonicated for 1 h. Then 200 μL of the ink was drop-casted on the glassy carbon working elctrode and subsequently dried naturally overnight. The loading on of the electrode was 0.4 mg/cm².

### Electrochemical Measurements

The electrochemical performance was carried out in a customized glass H-cell as previously reported. A platinum foil and Ag/AgCl were used as the counter and reference electrode, respectively. The working electrode was separate from the counter electrode by the Nafion-117 membrane (Fuel Cell Store). Before using, the Ag/AgCl reference was calibrated as reported. All potentials in this study were converted to the reversible hydrogen electrode (RHE) according to the Nernst equation (Evs.RHE=Evs.Ag/AgCl+0.231+0.0592×pH). The 10 mL of 0.5 M KHCO₃ solution electrolyte was added into the working and counter compartment, respectively. The cell was purged with high-purity CO₂ gas (Linde, 99.999%, 20 sccm) for 30 min prior to and throughout the duration of all electrochemical measurements. The electrochemical measurements were controlled and recorded with a CHI 650E potentiostat. The automatic iR (85%) compensation was used. The pH values of CO₂ and N₂ saturated 0.5 M KHCO₃ electrolyte were 7.25 and 8.36, which was detected by a pH meter (HI 2211, Hanna instruments). Gas-phase products were quantified by an on-line gas chromatograph (Ruimin GC 2060, Shanghai) equipped with a methanizer, a Hayesep-D capillary column, flame ionization detector (FID) for CO and thermal conductivity detector (TCD) for H₂. The CO₂ flow rate was controlled at 3 sccm using a standard series mass flow controller (Alicat Scientific mc-50 sccm). Each run was 8 min long. GC was calibrated using standard mixture gas (Linde) and diluted with nitrogen (Linde 99.999%). The liquid products were quantified after electrocatalysis using 1H NMR spectroscopy with solvent (H₂O) suppression. 450 μl of electrolyte was mixed with 500 μl of a solution of 10 mM dimethyl sulfoxide (DMSO) and in D₂O as internal standards for the 1H NMR analysis. The concentration of MeOH was calculated using the ratio of the area of the MeOH peak (at a chemical shift of 3.31 ppm) to that of the DMSO internal standard.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. The term “substantially coplanar” may refer to two surfaces within a few micrometers (μm) positioned along the same plane, for example, within 10 μm, within 5 μ, within 1 μm, or within 0.5 μm located along the same plane. When reference is made to “substantially” the same numerical value or characteristic, the term may refer to a value within ±10%, ±5%, ±1%, or ±0.5% of the average of the values.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A method of altering a degree of curvature of a molecular catalyst for CO₂ reduction reaction (CO₂RR) for higher catalytic activity, comprising:
providing a single-walled carbon nanotube (SWCNT);
providing a molecular catalyst having active sites for CO₂RR;
dispersing the molecular catalyst on the SWCNT; and
inducing a curvature of the active sites of the molecular catalyst.

2. The method of claim 1, wherein the dispersing the molecular catalyst on the SWCNT comprises:
providing a solution having N,N-dimethylformamide;
adding the molecular catalyst and the SWCNT into the solution;
performing a sonication to the solution; and
performing a magnetic stirring to the solution.

3. The method of claim 1, wherein the inducing the curvature of the active sites of the molecular catalyst comprises:
initiating a non-parallel π-π it interactions between the molecular catalyst and the SWCNT.

4. The method of claim 1, wherein the inducing the curvature of the active sites of the molecular catalyst comprises:
bending the active sites of the molecular catalyst from a flat configuration to a curved configuration.

5. The method of claim 1, wherein after the inducing the curvature of the active sites of the molecular catalyst further comprising:
receiving CO₂ at the active site;
releasing CO from the active site; and
releasing methanol from the active sites.

6. The method of claim 1, wherein the SWCNT has a diameter of 1-6 nm.

7. The method of claim 1, wherein the molecular catalyst is a macrocyclic molecule selected from a metal phthalocyanine, a metal porphyrin, a metal tetraphenylporphyrin, or a metal quaterpyridine.

8. The method of claim 7, wherein the metal comprises cobalt (Co), nickel (Ni), manganese (Mn), and iron (Fe).

9. The method of claim 7, wherein the molecular catalyst is a cobalt phthalocyanine or a nickel phthalocyanine.

10. A method of altering degree of curvature of a molecular catalyst comprising:
providing a single-walled carbon nanotube (SWCNT);
providing a molecular catalyst;
dispersing the molecular catalyst on the SWCNT; and
15 inducing a curvature of the active sites of the molecular catalyst.

11. The method of claim 10, wherein the molecular catalyst is a macrocyclic molecule selected from a metal phthalocyanine, a metal porphyrin, a metal tetraphenylporphyrin, or a metal quaterpyridine.

12. The method of claim 11, wherein the metal comprises cobalt (Co), nickel (Ni), manganese (Mn), and iron (Fe).

13. The method of claim 11, wherein the molecular catalyst is an iron phthalocyanine.

14. The method of claim 11, wherein after inducing the curvature of the active sites of the molecular catalyst further comprises:
effecting an oxygen reduction reaction by the molecular catalyst.

15. The method of claim 14, wherein the oxygen reduction reaction is a four-electron process.

16. The method of claim 10, wherein the inducing the curvature of the active sites of the molecular catalyst comprises:
bending the active sites of the molecular catalyst from a flat configuration to a curved configuration.

17. A molecular catalyst having an altered degree of curvature, wherein the molecular catalyst is dispersed on a single-walled carbon nanotube (SWCNT) for altering degree of curvature.

18. The molecular catalyst of claim 17, wherein the molecular catalyst having active sites for CO₂ reduction reaction (CO₂RR).

19. The molecular catalyst of claim 17, wherein the SWCNT has a diameter of 1-6 nm.

20. The molecular catalyst of claim 17, wherein the altered degree of curvature ranges from 1 to 96 degree.

21. A fuel cell with the molecular catalyst of claim 17, wherein the molecular catalysts having active sites for oxygen reduction reaction.
